(19) United States
(12) Patent Application Publication
(10) Pub. No.: US 2009/0258074 A1
(43) Pub. Date: Oct. 15, 2009

(54) MIXTURES OF TANNINS, THEIR PRODUCTION AND USE IN MEDICAMENTS OR AS DISINFECTANTS

(75) Inventors: Stephan Hüffer, Ludwigshafen (DE); Oliver Reese, Lemförde (DE); Günter Scherr, Ludwigshafen (DE); Sebastien Garnier, Weinheim (DE); Ulrich Mrowietz, Kronshagen (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/301,118

(22) PCT Filed: Feb. 28, 2007
(86) PCT No.: PCT/EP07/51884

§ 371 Date: Dec. 2, 2008

(30) Foreign Application Priority Data
May 17, 2006 (EP) 06114062.0

(51) Int. Cl. A61K 9/50; A61K 9/12; A61Q 11/00; A61K 8/18; A61Q 17/04

(52) U.S. Cl. 424/490; 424/45; 424/49; 424/59

(57) ABSTRACT

The invention relates to a mixture comprising
i) at least one condensation product (A) obtainable by reaction of
   a1) at least one aromatic system or heteroaromatic system,
   a2) at least one carbonyl compound,
   a3) if appropriate at least one sulfonating agent and
   a4) if appropriate at least one urea derivative,
      where the condensation product (A) has an M_{w} value ≧9000 g/mol, and
ii) at least one tanning agent having an M_{w} value ≦3000 g/mol.

The invention relates to a mixture comprising at least one condensation product (A), as defined below, having an M_{w} value ≧9000 g/mol and at least one tanning agent having an M_{w} value ≦3000 g/mol, processes for the production of such a mixture, its use as a medicament, and the pharmaceutical compositions comprising such a mixture. A further subject of the present invention is the use of the mixture as a disinfectant, for example in animal stables.

Tanning agents can in principle be divided into three main classes (see Römpps Chemie Lexikon [Römpp's Chemical Encyclopedia], 9th edition (1995), Georg Thieme Verlag Stuttgart, keyword “tanning agents”, pages 1541 to 1542):

1. inorganic tanning agents such as chromium(III) salts or polyphosphates; 2. synthetic organic tanning agents, which are usually obtainable by sulfonation of solubilized aldehyde condensation products of aromatic parent substances, in particular of phenol, cresol, naphthalene and naphthol; and 3. tanning agents of plant origin such as occur in leaves (tea), seeds (coffee), berries, galls or woods. In the narrower sense, tanning agents of plant origin are understood as meaning the “tannic acids” or “tannins”.

Both the tanning agents of plant origin (subsequently designated as plant or natural tanning agent) and the synthetic organic tanning agents (subsequently designated as synthetic tanning agent) are sometimes connected in the literature with antiviral action. This applies in particular to plant or synthetic tanning agents, which are designated as “polyphenols”.

For example, for plant tanning agents such as tannins an antiviral activity (in particular against herpes simplex) and antitumor activity of these natural tanning agents is described in T. Okuda, Phytochemistry, volume 66 (2005), pages 2012 to 2031 or Fukuji et al., Antiviral Res. 11 (1989), pages 285 to 298.

Furthermore, propolis, which is collected by bees from the buds, bark and wood of certain trees and contains plant tanning agents, is attributed, inter alia, an antiviral activity, for example against herpes simplex. Propolis, which is a complex mixture and contains, inter alia, polyphenol, can be composed, depending on the bee colony, of up to 200 different constituents, in particular these are chalcones, flavanones, flavones and flavanols (S. Bogdanov, Schweizerisches Zentrum für Bienenforschung [Swiss Center for bee research]; article obtainable from the Internet; http:www.apis.admin.ch/de/bienenprodukte/docs/produkte/propolis_d.pdf).

In the case of synthetic tanning agents too, pharmaceutical applications are already known. Thus, WO 95/14479 relates to a condensation polymer of aromatic sulfonic acids and an aldehyde for the inhibition of the HIV virus. It is described there that the higher the molecular weight of the polymer, the greater its therapeutic activity. Particularly preferably, condensation polymers having an M_{w} weight between 4000 and 12 000 g/mol are obtained by molecular size-dependent separation processes. In WO 95/14479, however, it is not disclosed that mixtures of synthetic tanning agents having a different molecular weight can also be used. The same applies analogously for U.S. Pat. No. 4,604,404, in which the use of sulfonated naphthalene-formaldehyde condensation polymers is described for the control of the herpes simplex virus. The polymers described therein have a molecular weight of preferably 2000 to 5000 g/mol.

Furthermore, DE-A 33 41 122 describes virucidal medicaments to be applied externally, in particular against herpes labilis and virus diseases of the skin. These medicaments are synthetic tanning agents, prepared by condensation of, for example, urea with phenol/cresol, formaldehyde and a sulfonating agent. No details with respect to the molecular weight of the polymers obtained here are found in DE-A 33 41 122.

In DE-A 10 2004 034613, condensation products are described which are obtainable by reaction of at least one aromatic system, at least one sulfonating agent, at least one carbonyl compound and if appropriate at least one urea derivative. Following the synthesis, the condensation products are subjected to at least one molecular size-dependent separation process. Here, the condensation product was separated into three fractions, a high molecular weight, a medium molecular weight and a low molecular weight fraction. It was found that the high molecular weight fractions have an improved activity with respect to the inhibition of the activity of the enzyme human leucocyte elastase compared to the corresponding medium molecular weight fractions of these condensation products. It is likewise not described in this document that mixtures of condensation products of different molecular weight can also be used as an antiviral medicament.

The German patent application having the number 10 2005 050 193.1 and EP-A 0 301 406 relate to synthetic tanning agents, in particular low molecular weight tanning agents, for which no use as medicaments is described.

The invention was therefore based on the object of making available further medicaments which are suitable as an antiviral agent; preferably these novel medicaments should have an improved action against viruses such as, for example, the herpes simplex virus. According to the invention, this object is achieved by a mixture comprising

i) at least one condensation product (A) obtainable by reaction of
a1) at least one aromatic system or heteroaromatic system,
a2) at least one carbonyl compound,
a3) if appropriate at least one sulfonating agent and
a4) if appropriate at least one urea derivative,
   where the condensation product (A) has an M_{w} value ≧9000 g/mol, and
   ii) at least one tanning agent having an M_{w} value ≦3000 g/mol.

One advantage of the present invention can be seen in that the mixtures according to the invention have improved activity as an antiviral agent, in particular with respect to the inhibition of the activity of the enzyme human leucocyte elastase. The inhibition of this enzyme is directly connected with an improved activity against the herpes simplex virus. The improved activity is achieved by mixing the high molecular weight fraction of the condensation product (A), that is the fraction which has an M_{w} value ≧9000 g/mol, with a tanning agent which can be described as of low molecular weight, that is has a molecular weight having an M_{w} value ≦3000 g/mol. Such mixtures surprisingly have a higher activity than the corresponding individual components at comparable concentration, based on the total concentration of the mixture according to the invention.

A further advantage of the mixtures according to the invention can be seen in that in the case in which a synthetic, formaldehyde-free tanning agent is employed as tanning agent having an M_{w} value ≦3000 g/mol, in particular using at least one condensation product (C) or (D), the proportion of formaldehyde-containing components in the mixture according to the invention can be decreased with at least constant activity. This is to be seen against the background that formaldehyde, which is a widespread starting material in the preparation of synthetic tanning agents, has in the meantime been classified as suspected of causing cancer by the World Health Organisation (WHO). Therefore, formaldehyde should be avoided as far as possible in the synthesis, since a certain residual formaldehyde content is always released in the condensation products obtained. Since, however, an apple, for example, also contains formaldehyde in low concentrations, accordingly low formaldehyde concentrations in pharmaceutical products are tolerable. As a result of these preferred embodiments of the mixtures according to the invention, the formaldehyde content, however, is reduced.

The individual mixture components are defined in more detail below.

### Condensation Product (A)

Condensation product (A) is obtainable by reaction of the following components:

a1) at least one aromatic system or heteroaromatic system

Aromatic systems are understood as meaning compounds having at least one phenyl ring, which can be substituted and which can also include a number of fused phenyl systems, for example naphthyl systems, phenanthrene systems and anthracene systems. If appropriate, in bi- or polycyclic systems individual cycles can also be completely or partly saturated, provided that at least one cycle is aromatic.

Heteroaromatic systems are described in the present invention as aromatic systems, which are preferably monocyclic or bicyclic, if appropriate also polycyclic, and contain at least one heteroatom, preferably selected from nitrogen, oxygen or sulfur. Examples of a heteroaromatic system are: pyrrole, furan, thiophene, imidazole, pyrazole, 1,2,3-triazole, 1,2,4-triazole, 1,3-oxazole (=oxazole), 1,2-oxazole (=isoxazole), oxadiazole, 1,3-thiazole (=thiazole), 1,2-thiazole (=isothiazole), tetrazole, pyridine, pyridazine, pyrimidine, pyrazine, 1,2,3-triazine, 1,2,4-triazine, 1,3,5-triazine, 1,2,4,5-tetrazine, indazole, indole, benzothiophene, benzofuran, benzothiazole, benzimidazole, quinoline, isoquinoline, quinazoline, cinnoline, quinoxaline, phthalazine, thienothiophene, 1,8-naphthyridine, other naphthyridines, purine or pteridine. Provided they are not monocyclic systems, in the case of each of the aforementioned heteroaromatic systems also the saturated form (perhydro form) or the partly unsaturated form (for example the dihydro form or tetrahydro form) or the maximally unsaturated (nonaromatic) form are additionally included for the second ring, provided the respective forms are known and stable. In the present invention, the description heteroaromatic system thus also comprises, for example, bi- or polycycles in which (in the case of the bicyclic system) both rings are aromatic, and bicyclic systems in which only one ring is aromatic. Such examples for heteroaromatic systems are: 3H-indoline, 2(1H)-quinolinone, 4-oxo-1,4-dihydroquinoline, 2H-1-oxoisoquinoline, 1,2-dihydroquinoline, 3,4-dihydroquinoline, 1,2-dihydroisoquinolinyl, 3,4-dihydroisoquinoline, oxindolyl, 1,2,3,4-tetrahydroisoquinoline, 1,2,3,4-tetrahydroquinoline, 5,6-dihydroquinoline, 5,6-dihydroisoquinoline, 5,6,7,8-tetrahydroquinoline or 5,6,7,8-tetrahydroisoquinoline.

Preferably, at least one aromatic system or heteroaromatic system is selected from benzene, naphthalene, anthracene, aromatic alcohols, aromatic ethers and aromatic sulfones.

The aromatic or heteroaromatic system (component a1) can be unsubstituted or at least monosubstituted. If one or more substituents are present, these are independently of one another chosen from C₁-C₁₀-alkyl groups such as, for example, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particularly preferably C₁-C₄-alkyl such as methyl, ethyl, n-propyl, isopropyl, n-butyl, isobutyl, sec-butyl and tert-butyl,

C₂-C₁₀-alkenyl groups, in particular vinyl, 1-allyl, 3-allyl, 2-allyl, cis- or trans-2-butenyl, ω-butenyl,
C₆-C₁₄-aryl groups aryl, such as, for example, phenyl, 1-naphthyl, 2-naphthyl, 1-anthryl, 2-anthryl, 9-anthryl, 1-phenanthryl, 2-phenanthryl, 3-phenanthryl, 4-phenanthryl and 9-phenanthryl, preferably phenyl, 1-naphthyl and 2-naphthyl, particularly preferably phenyl,
or benzyl groups.

Examples of preferred aromatic systems or heteroaromatic systems are:

benzene, toluene, ortho-xylene, meta-xylene, para-xylene, ethylbenzene, cumene, para-methylcumene, biphenyl, 2-methylbiphenyl, 3-methylbiphenyl, 4-methylbiphenyl, bitolyl (4,4′-dimethylbiphenyl), para-terphenyl, indene, fluorene, methylindenes (isomer mixture), naphthalene, 1-methylnaphthalene, 2-methylnaphthalene, 1,8-dimethylnaphthalene, 2,7-dimethylnaphthalene, phenanthrene, anthracene, 9-methylanthracene, 9-phenylanthracene.

Examples of aromatic alcohols which may be mentioned are: phenol, ortho-cresol, meta-cresol, para-cresol, 2-ethylphenol, 3-ethylphenol, 4-ethylphenol, 2,3-dimethylphenol, 2,4-dimethylphenol, 2,5-dimethylphenol, 2,6-dimethylphenol, 3,4-dimethylphenol, 3,5-dimethylphenol, gallic acid, α-naphthol, β-naphthol, 9-hydroxyanthracene as a tautomer of anthrone, 9-hydroxyphenanthrene, diphenylmethane, phenyl-(2-methylphenyl)methane, phenylparatolylmethane, phenylmetatolylmethane.

Examples of aromatic ethers which may be mentioned are: diphenyl ether, di-ortho-tolyl ether, di-meta-tolyl ether and di-para-tolyl ether.

Examples of aromatic sulfones which may be mentioned are diphenylsulfone and dihydroxydiphenylsulfone, in particular 4,4′-dihydroxydiphenylsulfone.

Component a1) is particularly preferably phenol.

In one embodiment of the present invention, mixtures of at least 2 aromatic systems are employed as component a1), for example mixtures of naphthalene and phenol, naphthalene and cresol (isomer mixture), naphthalene and diphenyl ether, naphthalene and ditolyl ether or phenol and ditolyl ether.

a2) at least one carbonyl compound

selected from aldehydes and ketones, preferably containing at least one aldehyde such as formaldehyde, acetaldehyde or propionaldehyde and in particular containing formaldehyde. If it is desired to employ formaldehyde, it is preferred to employ formaldehyde in aqueous solution.

a3) if appropriate at least one sulfonating agent

Suitable sulfonating agents are, for example, sulfuric acid, in particular concentrated sulfuric acid, furthermore oleum having an SO₃ content of 1 to 30% by weight, furthermore chlorosulfonic acid and amidosulfonic acid. Concentrated sulfuric acid and oleum having an SO₃ content of 1 to 15% by weight are preferred.

a4) if appropriate at least one urea derivative

In principle, urea and all derivatives thereof are suitable as component a4). A urea derivative is preferred which carries at least one hydrogen atom on each nitrogen atom.

Particularly preferably, at least one urea derivative is chosen from compounds of the general formula (I)

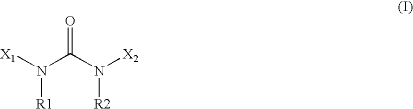

in which the variables are defined as follows:
X¹, X² are different or preferably identical and chosen from hydrogen and —CH₂OH,
R¹, R² are different or preferably identical and are chosen from hydrogen,
C₁-C₁₀-alkyl such as, for example, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particularly preferably C₁-C₄-alkyl such as methyl, ethyl, n-propyl, isopropyl, n-butyl, isobutyl, sec-butyl and tert-butyl, or
R¹ and R² together form a C₂-C₁₀-alkylene unit, unsubstituted or substituted by 2 to 5 hydroxyl groups, such as, for example, —(CH₂)₂—, —CH₂—CH(CH₃)—, —(CH₂)₃—, —CH₂—CH(C₂H₅)—, —(CH₂)₄—, —(CH₂)₅—, —(CH₂)₆—, —(CH₂)₇—, —(CH₂)₈—, —(CH₂)₉—, —(CH—OH)₂— (cis or trans), preferably C₂-C₄-alkylene; in particular —(CH₂)₂—, —(CH₂)₃—, and —(CH—OH)₂— (cis or trans).

(Unsubstituted) urea, melamine or the cyclic urea derivatives of the formulae I.1, I.2 or I.3 are very particularly preferred

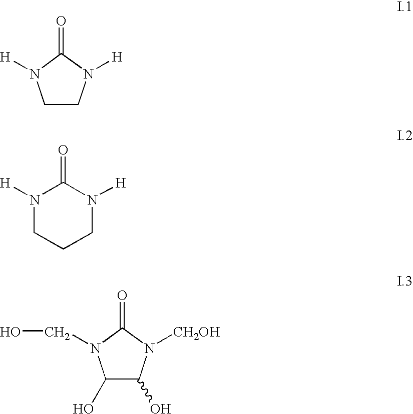

The condensation products (A) have an M_{w} value (weight-average molecular weight) ≧9000 g/mol, preferably of 10 000 to 100 000 g/mol, particularly preferably of 10 000-30 000. M_{w} values in the context of the present invention are determined by GPC standard procedures, BASF DIN standard 55672-1; solvent THF. Such methods are illustrated in more detail in the examples. Preferably, the ratio M_{w}/Mₙ here is <10, in particular M_{w}/Mₙ<5 (M_{w}=weight-average molecular weight, Mₙ=number-average molecular weight).

Processes for the preparation of a condensation product (A) are known to the person skilled in the art, for example they are described in EP-A 37 250, DE-A 1 113 457, Ullmann's Encyclopedia of Industrial Chemistry, volume A15, (5th edition) Weinheim 1990, pp. 259-282 or DE-A 848 823.

The reaction can be carried out in one or in a number of steps. For example, it is possible first
a1) to react at least one aromatic system or heteroaromatic system
a3) if appropriate with at least one sulfonating agent and then to react it in the same vessel without prior isolation with
a2) at least one carbonyl compound and
a4) if appropriate at least one urea derivative.

In another embodiment, it is possible to proceed by
a1) reacting at least one aromatic system or heteroaromatic system
a3) with at least one sulfonating agent, isolating the product and then reacting it with the reaction product of
a2) at least one carbonyl compound with
a4) at least one urea derivative.

It is possible in one embodiment of the present invention to react reactants a1) and a2) and if appropriate a3) and a4) in one portion in each case.

In another embodiment of the present invention, at least one reactant a1) to a4) is reacted in at least two portions.

In a special embodiment of the present invention, a number of reactants a1) and a2) and if appropriate a3) and a4) are reacted in a number of portions.

In one embodiment of the present invention, during the reaction
a5) one or more further reactants can be added, for example NaHSO₃, Na₂S₂O₅, KHSO₃, K₂S₂O₅, aqueous alkali metal hydroxide solution, in particular aqueous sodium hydroxide solution and aqueous potassium hydroxide solution, and aqueous ammonia. The reactant a5) serves in particular for the adjustment of the pH and the control of the solubility of the final product.

In one embodiment of the present invention, a1) to a5) reactants are chosen in the following ratio:
a1) the aromatic system(s) in the range from altogether 10 to 70% by weight, preferably altogether 20 to 60% by weight, particularly preferably altogether 35 to 50% by weight,
a2) the aldehyde(s) or the ketone(s) in the range from altogether 5 to 40% by weight, preferably altogether 10 to 30% by weight, particularly preferably altogether 15 to 25% by weight,
a3) if appropriate the sulfonating agent(s) in the range from altogether 5 to 50% by weight, preferably altogether 10 to 40% by weight, particularly preferably altogether 20 to 30% by weight, sulfonating agents always being calculated as SO₃,
a4) the urea derivative(s) in the range from 0 to altogether 30% by weight, preferably altogether 10 to 25 and particularly preferably 15 to 25% by weight,
   where % by weight are in each case based on the sum of all reactants a1) and a2), if appropriate a1) to a4),
a5) the additional reactant(s) in the range from 0 to altogether 30% by weight, preferably to altogether 25% by weight and particularly preferably altogether to 20% by weight,
   where the % by weight data from a5) are based on the sum of the reactants a1) and a2), if appropriate a1) to a4).

It is possible, for example, to react at temperatures in the range from 40 to 200° C., preferably 50 to 110° C. Customarily, the temperature of the reaction is adapted to a1) and a2). If it is desired, for example, to react aromatic alcohols, it is preferred to react at temperatures in the range from 50 to 110° C. Of course, it is also possible to set a certain temperature profile during the reaction. Thus it is possible, for example, first to start the reaction at 90 to 100° C. and after some time, for example, after 2 to 10 hours, to cool to 40 to 75° C. and to complete the reaction over a period of, for example, 1 to 10 hours.

Reaction is carried out, for example, at atmospheric pressure, but can, if desired, also be carried out at higher pressures, for example, 1.1 to 10 bar.

By means of the reaction described above, reaction solutions are obtained which customarily contain large amounts of acids such as, in particular, sulfuric acid or—in the case of the use of chlorosulfonic acid—HCl. Furthermore, reaction solutions can contain large amounts of alkali metal sulfate and/or alkali metal chloride.

Following the reaction described above, it is possible using, for example, aqueous alkali metal hydroxide solution or aqueous ammonia to set a pH in the range from 3 to 10, preferably 3.5 to 9.

By addition of water to reaction solutions obtainable by the reaction described above, it is possible by diluting with water to set a water content in the range from 70 to 95% by weight, preferably 75 to 90% by weight.

Consequent to the actual reaction and optionally consequent to the dilution with water, the reaction mixture obtainable by the reaction or the reaction solution obtainable by the reaction described above can be treated by molecular size-dependent separation processes. It is possible here to use one or more different molecular size-dependent separation processes or to carry out a molecular size-dependent separation process once or repeatedly. The use of a molecular size-dependent separation process is necessary if the condensation product (A) obtained by reaction of the individual components (a1) to a4)) has no M_{w} value ≧9000 g/mol. This means the condensation product (A) having an M_{w} value ≧9000 g/mol is isolated following the synthesis of the other synthesis products, for example fractions of condensation product (A) having a low M_{w} value or byproducts. Such a separation process can also be used in order to increase the M_{w} value of a given condensation product (A), which is ≧9000 g/mol, even further. It is known to the person skilled in the art, however, how the preparation of the condensation product (A) is to be controlled in order to obtain condensation products (A) having high M_{w} values, such that the carrying out of molecular size-dependent separation processes is not obligatory.

In a preferred embodiment of the present invention, in the preparation of the condensation product (A), after reaction of components a1) and a2) and if appropriate a3) and a4) a molecular size-dependent separation process, preferably an ultrafiltration, is carried out with obtainment of the condensation product (A) having an M_{w} value ≧9000 g/mol, preferably having an M_{w} value of 10 000 to 100 000 g/mol.

Suitable molecular size-dependent separation processes are, for example: preparative gel permeation chromatography and membrane separation processes such as, for example, microfiltration, nanofiltration and in particular ultrafiltration. Combinations of microfiltration and ultrafiltration are also suitable. Microfiltrations and ultrafiltrations and membranes necessary therefor are known as such and described, for example, in Ullmann's Encyclopedia of Industrial Chemistry, 6th edition, vol. 21, Wiley-VCH Weinheim, pp. 243-321. Nanofiltrations and the membranes necessary for this are likewise known as such and described in R. Rautenbach, “Membranverfahren” [Membrane Processes], Springer Verlag Berlin Heidelberg 1997.

Ultrafiltrations are known as such and are in general operated as crossflow ultrafiltrations. Suitable membranes are commercially available membranes which are prepared, for example, from organic materials such as polysulfones or polyvinylidene fluoride or preferably from inorganic materials such as, for example, TiO₂, ZrO₂ or Al₂O₃. Customary forms are capillary, tubular and flat membranes, the latter in the form of membrane pads or spirally wound modules.

For example, a transmembrane pressure difference, i.e. a pressure difference between feed and permeate, in the range from 1 to 200 bar, preferably in the range from 1.2 to 100 bar, is used in membrane separation processes and in particular in ultrafiltrations.

In one embodiment, the temperature of the treated reaction solution after membrane separation processes is in the range from 20 to 70° C., preferably 25 to 35° C.

In one embodiment of the present invention, at least one membrane having a molecular weight cut-off in the region of 1000 daltons, preferably 2000 daltons, particularly preferably 5000 daltons, very particularly preferably 7500 daltons and even more preferably of 15 000 daltons is employed. The molecular weight cut-off is also designated as a separation limit.

In one embodiment of the present invention, the ultrafiltration is carried out such that a certain mass ratio of permeate to retentate is established at the end of the ultrafiltration. The amount of retentate customarily remains constant during the ultrafiltration as a result of continuous readdition of water, the amount of permeate increases in the course of the filtration period. Customary values lie in the range from 0.5:1 to 10:1, preferably 0.8:1 to 5:1, particularly preferably 1.0:1 to 3:1.

Visually essentially transparent aqueous solutions of condensation products (A) are customarily obtained.

It is possible to isolate the condensation products (A) from the solutions described above, for example by evaporating the water or by spray drying.

In one embodiment of the present invention, the condensation products (A) have a salt content of inorganic salts such as, for example, alkali metal sulfate and alkali metal chloride of 10 ppm to less than 5% by weight, preferably less than 2% by weight, particularly preferably less than 1% by weight and very particularly preferably less than 0.5% by weight, based on the dry weight of condensation product (A). The salt content can be determined, for example, by ion chromatography (IC), as described, for example, in Römpps Lexikon Chemie [Römpp's Chemical Encyclopedia], 10th edition, Georg Thieme Verlag Stuttgart New York, volume 2, keyword: ion chromatography.

In one embodiment of the present invention, the condensation products (A) have a residual monomer content of 10 ppm to less than 5% by weight, preferably less than 2% by weight, based on the dry weight of the condensation product (A). Residual monomer in the context of the present invention is designated as reactants a), c) and d) not reacted to completion which can be found in condensation products (A). The residual monomer content can be determined, for example, by gel permeation chromatography (GPC) or preferably by ion chromatography (IC) or high pressure liquid chromatography (HPLC).

In one embodiment of the present invention, condensation products according to the invention have a content of free carbonyl compound a2) including carbonyl compound present as hydrate a2) in the range from 1 ppm to less than 0.5% by weight, preferably 0.1% by weight or less, based on the dry weight of condensation product according to the invention. In this embodiment, the amount of free carbonyl compound a2), of course, relates to the carbonyl compound a2) which has been employed in the reaction of a1) and a2) and if appropriate a2) and a4). If a number of carbonyl compounds a2) have been employed, the content of free carbonyl compound a2) relates to the sum of all of the carbonyl compounds a2) which has been employed in the reaction of a1) and a2) and if appropriate a3) and a4). The determination of the content of free carbonyl compound a2) can be carried out by methods known per se. If carbonyl compound a2) is a solid or liquid at room temperature, the content of the free carbonyl compound a2) can be determined, for example, by gas chromatography or HPLC. If carbonyl compound a2) is formaldehyde, it can be determined, for example, photometrically. A particularly preferred method for the determination of free formaldehyde is the reaction with acetylacetone and ammonium acetate to give diacetyldihydrolutidine and photometric measurement of diacetyldihydrolutidine at a wavelength of 412 nm.

Tanning Agent Having an M_{w} Value ≦3000 g/mol

Suitable tanning agents having an M_{w} value ≦3000 g/mol are in principle all tanning agents which have an appropriate M_{w} value. Preferably, the M_{w} value lies between 300 and 3000 g/mol; an M_{w} value between 300 and 3000 g/mol is particularly preferred, where the ratio M_{w}/Mₙ is <10 (M_{w}=weight-average molecular weight, Mₙ=number-average molecular weight), in particular M_{w}/Mₙ is <5.

Tanning agents having an M_{w} value ≦3000 g/mol can be either an inorganic tanning agent, a plant tanning agent or a synthetic tanning agent (for this see the abovementioned definition according to Römpps Chemie Lexikon, 9th edition (1995), Georg Thieme Verlag, Stuttgart, keyword: “tanning agents”, pages 1541 to 1542). Preferably, natural or synthetic tanning agents are used as tanning agents having an M_{w} value ≦3000 g/mol, synthetic tanning agents are particularly preferably preferred here.

In the mixtures according to the present invention, the component (i) [at least one condensation product (A)] and the component (ii) [at least one tanning agent having a M_{w}-value ≦3000 g/mol] can be present at each ratio. The component (i) is present preferably at 10-90 percent by weight (wt.-%), more preferably at 30-70 wt.-%, most preferably at 40-60 wt.-%, and component (ii) at 10-90 wt.-%, more preferably at 30-70 wt.-%, most preferably at 40-60 wt.-% within the mixtures according to the present invention. Said wt.-% values relate to the sum of the components (i) and (ii) contained within the mixture.

Additionally, the mixtures according to the present invention can contain further components such as water or further polymers/condensation products, which do not fall under the definition of components (i) and (ii).

In one embodiment of the present invention, the sum of the components (i) and (ii) is at least 90 wt.-%, preferably at least 95 wt.-%, especially at least 99 wt.-% related to the condensation products and/or tanning agents contained in the mixture.

Examples of plant tanning agents are tannins such as catechols or gallic acid derivatives such as gallates. Plant tanning agents which are based on gallic acid derivatives (such as gallates) differ from the condensation products according to the invention in particular in that the last-mentioned have in their chemical structures (a multiplicity of) —CR¹R² bridges (crosslinkages), which are derived from the carbonyl compound a2) employed and which are not present in plant tanning agents. If, for example, formaldehyde is employed as component a2), the condensation products have —CH₂ bridges. Plant tanning agents (gallates) are typically oligomeric systems, whereas the condensation products according to the present invention are preferably polymers.

Preferred plant tanning agents are tannins from the group consisting of the catechols, epicatechols and epigallocatechols and their gallates.

Tannin is understood in principle as meaning naturally occurring polyphenols, such as are mentioned, for example, in T. Okuda, Phytochemistry, volume 66 (2005), pages 2012 to 2031 or Römpp's Chemie Lexikon, 9th edition (1995), Georg Thieme Verlag, Stuttgart, keyword “tannins”, pages 4452 to 4453. Preferred tannins are ellagitannins and dehydroellagitannins, in particular geraniin, dehydrogeraniin, furosinin, ascorgeraniin, geraniinic acid, mallotusinic acid, pentagalloylglucose, camelliatannin A, casuariin, euphorbin E, camelliatannin F, agrimoniin, trapanin B, oenothein A, oenothein B or gemin D, lignin and ligninsulfonates. Catechols, epicatechols and epigallocatechols are furthermore preferred.

Examples of a suitable catechol or derivatives thereof in particular comprise flavan-3-ols, flavan-3,4-diols (leucoanthocyanidins) and flavanones, flavones, chalcones or dihydrocychalcones, epicatechols and epigallocatechols.

Examples of suitable gallic acid derivatives are mentioned, for example, in H. Sakagami et al, Anticancer Research 17 (1997), pages 377 to 380. Preferably, these are gallic acid, methyl tri-O-methylgallate, tri-O-methylgallic acid, methyl tri-O-acetylgallate, methyl gallate, ethyl gallate, n-propyl gallate, isoamyl gallate, lauryl gallate, stearyl gallate, epigallocatechol gallate and gallic acid.

For example, extracts of green tea can also be employed as plant tanning agents, like-wise extracts of chestnuts or mimosa.

Synthetic tanning agents as such and processes for the preparation are known to the person skilled in the art. Suitable synthetic tanning agents having an M_{w} value ≦3000 g/mol are disclosed, for example, in EP-A 0 301 406 or DE-A 10 2005 050 193.1. Methods using which the molar mass can be controlled in a certain range by control of the synthesis parameters are known to the person skilled in the art.

Preferably, the mixtures according to the invention contain as a synthetic tanning agent having an M_{w} value ≦3000 g/mol at least one of the condensation products (B) to (D) mentioned below.

### Condensation Product (B)

Condensation product (B) is obtainable by reaction of
b1) at least one aromatic system or heteroaromatic system,
b2) at least one carbonyl compound,
b3) if appropriate at least one sulfonating agent and
b4) if appropriate at least one urea derivative,
   where the condensation product (B) has an M_{w} value between 300 and 3000 g/mol. An M_{w} value between 300 and 3000 g/mol is particularly preferred, where the ratio M_{w}/Mₙ is <10 (M_{w}=weight-average molecular weight, Mₙ=number-average molecular weight), in particular M_{w}/Mₙ is <5.

The components b1) to b4) correspond, including the preferred definitions, to the components a1) to a4) of the condensation product (A). Condensation product (A) differs, however, from condensation product (B) by the M_{w} value. Furthermore, in contrast to component a1), other than phenol dihydroxydiphenylsulfone, in particular 4,4′-dihydroxydiphenylsulfone, is also a particularly preferred component b1).

Processes for the preparation of condensation products (B) having a low M_{w} value (M_{w} value ≦3000 g/mol) are known to the person skilled in the art. Such condensation products can be prepared specifically, in particular by influencing parameters such as reaction time, temperature (rather lower), the choice of the monomer (influences the reactivity, in particular use of dihydroxydiphenylsulfones) or pH (weakly acidic). Alternatively, condensation products (B) can also be prepared by carrying out a molecular size-dependent separation process, preferably an ultrafiltration where the condensation product (B) is isolated from all other constituents—as described for condensation product (A)—following the synthesis of an appropriate condensation product. Condensation products (B) having the desired M_{w} value can in particular be separated and isolated by use of a membrane having a suitable molecular weight cut-off range of 1000 D-2500 D.

### Condensation Product (C)

Condensation product (C) is obtainable by reaction of
c1) melamine and/or urea,
c2) glyoxal, glyoxylic acid or an alkali metal salt thereof,
c3) if appropriate at least one aromatic compound having at least one phenolic hydroxyl group and
c4) if appropriate at least one condensable compound having a reactive nitrogen-containing group,
   where the condensation product (C) has an M_{w} value between 300 and 3000 g/mol. An M_{w} value between 300 and 3000 g/mol is particularly preferred, where the ratio M_{w}/Mₙ is <10 (M_{w}=weight-average molecular weight, Mₙ=number-average molecular weight), in particular M_{w}/Mₙ is <5.

The condensation products (C) as such and processes for their preparation are known to the person skilled in the art. For example, these are described in EP-A 0 301 406 and are additionally included by reference in the present invention.

Suitable components c3) are, for example, phenolsulfonic acid, sulfosalicylic acid, salicylic acid and 8-hydroxyquinoline 4,4′-dihydroxydiphenylsulfone. Suitable components c4) are carboxylic acid amides, sulfonic acid amides, imides, ureas, amino and imino acids and dialkylamines and dialkanolamines. Examples thereof are acetamide, benzamide, formamide, amidosulfonic acid, succinimide, glycine, iminodiacetic acid, phenylglycine, urea, dicyandiamide, diethanolamine or diethylamine. Acidic compounds can be condensed here in the form of their alkali metal salts. Acetamide and amidosulfonic acid are particularly preferred as component c4).

A preferred condensation product (C) is obtainable by reaction of
c1) melamine and/or urea,
c2) glyoxal and/or glyoxylic acid and
c4) if appropriate amidosulfonic acid.

### Condensation Product (D)

Condensation product (D) is obtainable by reaction of
d1) at least one cyclic organic carbonate with
d2) at least one compound having at least two nucleophilic groups per molecule, chosen from sulfonic acid groups, hydroxyl groups, primary or secondary amino groups or mercapto groups,
   where the condensation product (D) has an M_{w} value between 300 and 3000 g/mol. An M_{w} value between 300 and 3000 g/mol is particularly preferred, where the ratio M_{w}/Mₙ is <10 (M_{w}=weight-average molecular weight, M_{w}=number-average molecular weight), in particular M_{w}/Mₙ is <5

Condensation products (D) as such and processes for their preparation are known to the person skilled in the art, they are disclosed, for example, in the German application having the number DE-A 10 2005 050 193.1 and are included by reference in the present invention.

Cyclic organic carbonates (component d1) are understood in the context of the present invention as meaning organic carboxylic acid esters which contain at least one cyclic group.

Preferably, cyclic organic carbonates are those organic carboxylic acid esters in which the carboxylic acid ester group is part of a cyclic system.

In one embodiment of the present invention the cyclic organic carbonate (d1) is chosen from compounds of the general formula (II)

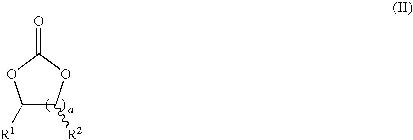

where the variables are defined as follows:
R¹ chosen from C₁-C₄-alkyl, branched or preferably linear, for example, methyl, ethyl, n-propyl, isopropyl, n-butyl, isobutyl, sec-butyl, tert-butyl, very preferably methyl and ethyl, and very particularly preferably hydrogen,
R² if appropriate different or preferably identical and independently of one another chosen from hydrogen and C₁-C₄-alkyl, branched or preferably linear, for example, methyl, ethyl, n-propyl, isopropyl, n-butyl, isobutyl, sec-butyl, tert-butyl, very preferably methyl and ethyl, and very particularly preferably in each case identical and hydrogen,
a an integer in the range from 1 to 3, preferably 2 and particularly preferably 1.

Particularly preferred cyclic organic carbonates d1) are propylene carbonate or ethylene carbonate. Mixtures of propylene carbonate (R¹=methyl, R²=hydrogen, a=1) and ethylene carbonate (R¹═R²=hydrogen, a=1), in particular mixtures of propylene carbonate and ethylene carbonate which are liquid at room temperature are likewise particularly preferred.

Component d2) is understood as meaning those compounds which contain two groups capable of nucleophilic reactions such as, for example, sulfonic acid groups, hydroxyl groups, mercapto groups or primary or secondary amino groups.

Examples of suitable compounds d2) can contain:

at least two nucleophilic hydroxyl groups per molecule,
at least two nucleophilic mercapto groups per molecule,
at least two nucleophilic primary or secondary amino groups per molecule, for example, two or three nucleophilic primary or secondary amino groups per molecule,
at least one nucleophilic hydroxyl group or mercapto group and at least one nucleophilic primary or secondary amino group per molecule or
at least one nucleophilic hydroxyl group and at least one nucleophilic mercapto group per molecule,
at least one nucleophilic hydroxyl group or primary or secondary amino group and one sulfonic acid group per molecule.

Sulfuric acid is not a compound d2) within the meaning of the present invention.

Examples of nucleophilic hydroxyl groups are OH groups of primary and secondary alcohols and in particular phenolic OH groups.

Examples of nucleophilic mercapto groups are SH groups, aliphatic or aromatic.

Examples of nucleophilic amino groups are —NHR³ groups, aliphatic or aromatic, where R³ is chosen from hydrogen, C₁-C₄-alkyl, as defined above, and CN, or the NH₂ group of, for example, amidosulfonic acid.

OH groups and NH groups, which are constituents of aminal groups, hemiaminal groups or hydrate groups of ketones or aldehydes, are not nucleophilic hydroxyl groups or amino groups within the meaning of the present invention. OH groups and NH groups which are constituents of carboxylic acid groups or carboxylic acid amide groups are likewise not nucleophilic hydroxyl groups or amino groups within the meaning of the present invention.

Preferred examples of compounds d2) are
i) ureas, unsubstituted or mono- or di-N,N′-substituted by C₁-C₄-alkyl, biuret, in particular unsubstituted urea,
ii) heterocyclic compounds having at least two NH₂ groups per molecule, for example adenine and in particular melamine,
iii) benzoguanamine, dicyandiamide, guanidine,
iv) compounds of the general formula (III)

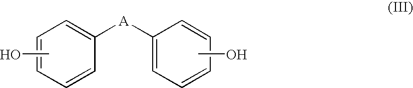

in which A is a bivalent group, for example —CH₂—, —CH₂CH₂—, —CH(CH₃)—, —C(CH₃)₂—, —CO—, —SO₂—, preferably 4,4′-dihydroxybiphenyl, 2,4′-dihydroxy-diphenylsulfone, particularly preferably 4,4′-dihydroxydiphenylsulfone, mixtures of 4,4′-dihydroxydiphenylsulfone and 2,4′-dihydroxydiphenylsulfone, for example, in a weight ratio of 8:1 to 8:1.5, and bisphenol A.

Further preferred examples of compound d2) are 4-hydroxyphenylsulfonic acid and amidosulfonic acid.

Particularly preferred compounds d2) are selected from melamine, biuret, dicyanamide, amidosulfonic acid and 4,4′-dihydroxydiphenylsulfone.

In a preferred embodiment of the present invention, mixtures are employed in which at least one condensation product (A) and/or at least one synthetic tanning agent having an M_{w} value ≦3000 g/mol are prepared using at least one compound which contains at least one hydroxyl group or is substituted by such a group. Preferably, this is achieved by

the component a1) containing at least one compound which is substituted by at least one hydroxyl group and/or
the component b1) containing at least one compound which is substituted by at least one hydroxyl group and/or
the component c3) being present and/or
the component d2) containing at least one compound having at least one hydroxyl group as a nucleophilic group.

In a further preferred embodiment of the present invention, mixtures are employed in which the tanning agent having an M_{w} value ≦3000 g/mol is formaldehyde-free, preferably a synthetic formaldehyde-free tanning agent. Preferably, this is achieved by a condensation product (C) or condensation product (D) being employed in the mixture.

A further subject of the present invention relates to a process for the preparation of the mixture according to the invention. The process comprises preparing the individual constituents of the mixture (at least one condensation product (A) and at least one tanning agent having an M_{w} value ≦3000 g/mol) separately by reaction of the respective starting materials and if appropriate subjecting them to a molecular size-dependent separation process, whereupon the individual components are mixed to give the mixture. In an alternative embodiment, the mixtures according to the invention which contain at least one condensation product (A) and at least one condensation product (B) are prepared by preparing these two condensation products together and subsequently isolating them by a molecular size-dependent separation process from the other products or byproducts which are obtained in the preparation process. Following this, the two isolated condensation products (A) and (B) are mixed to give the mixture according to the invention. If appropriate, further condensation products (A) or (B) or further tanning agents having an M_{w} value ≦3000 g/mol can also be admixed.

The present invention also relates to the use of one of the mixtures described above comprising at least one condensation product (A) having an M_{w} value ≧9000 g/mol and at least one tanning agent having an M_{w} value ≦3000 g/mol as a medicament.

The condensation products according to the invention are suitable, in particular, as an antiviral agent, that is as drugs against viruses, also called virustatics or virucidal agents. Preferably, they are suitable as an antiviral agent against human papillomaviruses, especially type 16, 18, 6 and 11, endogenous retroviruses, in particular the HERV type (human endogenous retroviruses), herpes viruses, in particular HSV-1, HCMV viruses (human cytomegalovirus) or HIV viruses.

Furthermore, the condensation products according to the invention are preferably suitable as an antiviral agent against coronaviruses (e.g. SARS (severe acute respiratory syndrome)-associated coronavirus), flaviviruses (e.g. West Nile Virus (WNV)), togaviruses (e.g. Chikungunya virus) or paramyxoviruses (e.g. measles, respiratory syncytial virus (RSV)).

Preferably, the mixtures according to the invention (for the preparation of a medicament) are suitable for the prophylaxis and/or treatment of genital warts, cervical cancer, allergic or nonallergic eczemas, diaper rash, pruritus, inflammatory diseases, autoimmune diseases, in particular arthritis, of melanomatous carcinomas, inflammations of the skin, herpes, in particular herpes labilis and herpes simplex, chickenpox, herpes zoster, influenza or Aids (HIV).

In one embodiment of the present invention, medicaments are medicaments for the local treatment of allergic or nonallergic eczemas, diaper rash or pruritus.

In a special embodiment of the present invention, medicaments are medicaments for the treatment of inflammatory diseases of the skin, in which, as a result of enzymatic activity, e.g. of human leucocyte elastase, the formation of vesicles, pustules and “spongiosis” in the epidermis occurs. The medicaments are preferably applied externally.

In a preferred embodiment of the present invention, medicaments are medicaments against viruses, preferably retroviruses, for example RNA viruses (ribonucleic acid viruses) and DNA viruses (deoxyribonucleic acid viruses) and in particular herpes viruses, for example viruses which produce herpes simplex (HS viruses), or alternatively viruses which produce chickenpox and influenza. Furthermore, it is to be noted that the active compounds according to the invention can be employed both against hydrophilic and likewise against lipophilic/hydrophobic viruses.

In a further embodiment of the present invention, medicaments are medicaments against HIV viruses (human immunodeficiency virus). It is known of the HIV virus that it causes Aids (acquired immunodeficiency syndrome).

In a further preferred embodiment of the present invention, medicaments are medicaments against human papillomaviruses and endogenous retroviruses (HERV type). The human papillomaviruses are in particular the types 16, 18, 6 and 11. In this respect, the mixtures according to the invention are suitable, in particular, for the external medication of genital warts and cancer of the cervix. In connection with the treatment of HERV viruses (in particular HERV-K), the mixtures according to the invention are suitable for the treatment of autoimmune diseases (arthritis) and preventively against melanomatous carcinomas.

In the above embodiments, the term treatment also comprises the prophylaxis, therapy or cure of the aforementioned diseases.

The mixtures according to the invention can be administered to animals and humans, preferably mammals and humans, particularly preferably humans. The mixtures according to the invention can here be administered themselves as medicaments, as mixtures with one another or mixtures with other medicaments or in the form of pharmaceutical compositions. Consequently, the present invention likewise relates to the use of the mixtures according to the invention for the production of one or more medicaments for the prophylaxis and/or treatment of the aforementioned diseases or as antiviral agents, to pharmaceutical compositions comprising an efficacious amount of at least one mixture according to the invention and to the use of these pharmaceutical compositions for the prophylaxis and/or treatment of the aforementioned diseases.

The pharmaceutical compositions according to the invention comprise an efficacious amount of at least one mixture according to the invention and a physiologically tolerable vehicle. The pharmaceutical compositions can be present here in different administration forms, in particular in the form of a pill, tablet, lozenge, granules, capsule, hard or soft gelatin capsule, aqueous solution, alcoholic solution, oily solution, syrup, emulsion, suspension, suppository, pastille, solution for injection or infusion, ointment, tincture, cream, lotion, powder, spray, of a transdermal therapeutic system, nasal spray, aerosol, aerosol mixture, microcapsule, implant, rod, patch or gel. Likewise, the pharmaceutical composition according to the invention can also be a constituent of health care products such as sunscreen creams, nasal sprays, mouthwashes, toothpastes, plasters, (moist) wipes, washing lotions or shampoos.

Depending on the administration form used, the mixtures according to the invention are processed with physiologically tolerable vehicles which are known as such to the person skilled in the art to give the pharmaceutical compositions according to the invention. The vehicle, of course, must be tolerable in the sense that it is compatible with the other constituents of the composition and is not harmful to the health of the patient (physiologically tolerable). The vehicle can be a solid or a liquid or both and is preferably formulated with the compound as an individual dose, for example as a tablet which can contain 0.05 to 95% by weight of the active compound (mixture according to the invention). Further pharmaceutically active substances can likewise be present. The pharmaceutical compositions according to the invention can be prepared according to one of the known pharmaceutical methods, which essentially consists in mixing the constituents with pharmacologically tolerable vehicles and/or further excipients such as fillers, binders, lubricants, wetting agents, stabilizers et cetera.

Preferred pharmaceutical compositions in the context of the present invention are listed below.

In one embodiment of the present invention, ointments, creams, fatty creams, gels, lotions or powders according to the invention can in each case contain mixtures according to the invention in the range from 0.1 to 5% by weight, preferably 0.2 to 3% by weight, based on the respective ointment, cream, fatty cream, lotion or the respective gel or powder.

In one embodiment of the present invention, powders or concentrates according to the invention can contain mixture according to the invention in the range from 1 to 75% by weight, preferably 10 to 65% by weight, based on the respective powder or concentrate.

Creams according to the invention are customarily oil-in-water emulsions, ointments according to the invention are customarily water-in-oil emulsions. In addition to preferably purified water, ointments and creams according to the invention contain one or more oil components and preferably one or more surface-active substances, for example one or more emulsifiers or protective colloids. Furthermore, ointments and fatty creams according to the invention—as also other administration forms according to the invention of the condensation products according to the invention—can contain preservatives such as, for example, sorbic acid.

Suitable oil components are natural and synthetic waxes, natural and synthetic oils such as, for example, nut oil, fish oil, olive oil and polymers such as, for example, polyacrylic acid, polydimethylsiloxane and polymethylphenylsiloxane.

Suitable surface-active substances are, for example, compounds of the general formula (IV)

CH₃—(CH₂)ₙ—X—R³ (IV)

where the variables are defined as follows:
n is an integer in the range from 0 to 20, preferably an even number in the range from 2 to 16 and
X is double-bonded groups which carry at least one atom different from carbon and hydrogen, preferably nitrogen and particularly preferably oxygen, in particular —O— and —COO—,
R³ is chosen from
   hydrogen,
   C₁-C₁₀-alkyl groups such as, for example, methyl, ethyl, n-propyl, isopropyl, n-butyl, isobutyl, sec-butyl, tert-butyl, n-pentyl, isopentyl, sec-pentyl, neopentyl, 1,2-di-methylpropyl, isoamyl, n-hexyl, isohexyl, sec-hexyl, n-heptyl, n-octyl, 2-ethylhexyl, n-nonyl, n-decyl; particularly preferably C₁-C₄-alkyl such as methyl, ethyl, n-propyl, isopropyl, n-butyl, isobutyl, sec-butyl and tert-butyl,
   —(CH₂—CH₂—O)ₘ—H, where m is an integer in the range from 1 to 100, preferably to 25,
   CH₃—(CH₂)ₙ—X—(O—CH₂—CH₂)ₘ—, where X and n can in each case be different or preferably identical.

Furthermore, ointments and creams according to the invention—as also other administration forms according to the invention of the condensation products according to the invention—can contain organic solvents such as, for example, propylene glycol and glycerol.

Preferred examples of surface-active substances are, for example, isopropyl tetradecanoate, cetyl alcohol, palmitic acid, stearic acid, polyoxyethylene 2-stearyl ether, α-n-dodecyl-ω-hydroxypolyoxyethylene on average having 10 ethylene oxide units, 2-phenoxyethanol, polyoxyethylene 21-stearyl ether.

Fatty creams according to the invention are customarily water-in-oil emulsions and in addition to preferably purified water contain one or more oil components and preferably one or more surface-active substances, for example one or more emulsifiers or protective colloids.

In addition to the oil components described above, suitable oil components are natural and synthetic fats such as, for example, mono- or polyethylenically unsaturated fatty acid glycerides.

Furthermore, fatty creams according to the invention can contain one or more of the following substances: methyl 4-hydroxybenzoate, propyl 4-hydroxybenzoate, aqueous sorbitol solution, tris[4-n-dodecylpoly(oxoethylene)]phosphate, cetylstearyl alcohol, hexyl laurate, vitamin F glycerol ester, dimethicone 350, calcium lactate pentahydrate.

Gels according to the invention can contain, for example, polyacrylic acid, sodium hydroxide and butylhydroxyanisole, for example, 4-methoxy-2-tert-butylphenol, 4-methoxy-3-tert-butylphenol and mixtures of the two aforementioned compounds.

Lotions according to the invention can contain, for example, at least one of the substances mentioned below: glycerol, zinc oxide, talc, lecithin, highly disperse silica, isopropanol, methyl 4-hydroxybenzoate, carageenan, sodium salt and phosphoric acid ester of the general formula (V)

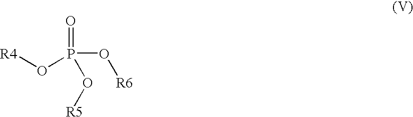

in which R⁴, R⁵ and R⁶ can be identical or different and are chosen from n-C₁₀-C₂₀-alkyl, in particular n-C₁₆-C₁₈-alkyl and H—(O—CH₂—CH₂)ₘ, where m is defined as above.

Powders according to the invention can contain, for example: calcium lactate pentahydrate, talc, maize starch, 2-n-octyl-1-dodecanol, silica.

Powders according to the invention for the preparation of solutions for use can contain, for example, calcium lactate 5H₂O and sodium sulfate (as a vehicle).

Concentrates according to the invention for the preparation of solutions for use can contain, for example: sodium salt of 2-dodecylpoly(oxyethylene) hydrogensulfate, sodium sulfate as a vehicle.

Instead of investigating ointments, creams, fatty creams, gels, lotions, cosmetic powders, powders or concentrates according to the invention for their efficacy, condensation products according to the invention, if appropriate as a stock solution, can be investigated for their efficacy. Suitable investigation methods are investigations on the inhibition of selected enzymes, for example human leucocyte elastase or the protease plasmin. Furthermore, it can be investigated to what extent the replication of viruses concerned is inhibited. Such investigation methods are described even more specifically in the following text (pharmacological investigations).

A further subject of the present invention is the use of the mixture according to the invention comprising at least one condensation product (A) and at least one tanning agent having an M_{w} value ≦3000 g/mol (as defined above) for disinfection, as a disinfectant or constituent of a disinfectant. In particular, the mixtures according to the invention are used in the hospital sector, in particular hospital intensive care units, toilets, washrooms, households, food production or in stables or cages of animals, in particular of birds, pigs and cattle.

The mixtures according to the invention are distinguished in their use as disinfectants in that they have a surprisingly good broad-spectrum action against fungi, bacteria and viruses and a lower toxicity compared to the customary agents or mixtures which are used as disinfectants according to the prior art. Furthermore, they are neither volatile nor irritating to the mucous membranes and they can be readily prepared both as a liquid or alternatively scatterable powder. In particular, the mixtures according to the invention are suitable for use in stables or cages of animals, preferably on straw.

A further subject of the present invention is thus also a disinfectant comprising at least one mixture according to the invention (as in the above definitions) comprising

i) at least one condensation product (A) obtainable by reaction of
a1) at least one aromatic system or heteroaromatic system,
a2) at least one carbonyl compound,
a3) if appropriate at least one sulfonating agent and
a4) if appropriate at least one urea derivative,
where the condensation product (A) has an M_{w} value ≧9000 g/mol, and
   ii) at least one tanning agent having an M_{w} value ≦3000 g/mol.

The disinfectants according to the invention are thus not intended for administration as medicaments, but they are suitable for the disinfection of, for example, the abovementioned articles. In the disinfectants according to the invention, at least one mixture according to the invention is present in the customary concentrations. Further components which are present in the disinfectants according to the invention are known to the person skilled in the art. Such components can vary depending on the field of application, the same applies for the concentration in the mixture according to the invention.

The invention will be illustrated by the following examples.

### EXAMPLES

### Synthesis Examples of Formaldehyde-Free Low Molecular Weight Tanning Agent

### Example NM1 Condensation Product (C)

### Reactants:

urea
glyoxal

33.0 g (549 mmol) of urea are dissolved in 180 ml of water in a flask and heated to 50° C. with stirring. 218 g (1.50 mol) of glyoxal solution (40%) are added at this temperature and the mixture is stirred for a further 30 min. After cooling to room temperature, it is adjusted to a pH of 5 using sodium hydroxide solution (50%). About 430 g of a clear solution are obtained having a solids content of 28% [M_{w}=2850 g/mol, M_{w}/Mₙ=11.3]

### Example NM2 Condensation Product (C)

### Reactants:

melamine
glyoxal

A mixture of 193.0 g of 40% strength aqueous glyoxal solution (1.33 mol) and 21.0 g of melamine (0.17 mol) are warmed to 40° C. for 15 min, a clear solution resulting. Subsequently, this is cooled and adjusted with 31.5 g of water to a solids content of calculated 40% [M_{w}=2640 g/mol, M_{w}/Mₙ=8.8].

### Example NM3 Condensation Product (D)

### Reactants:

melamine
ethylene carbonate
sulfuric acid

24.0 g (190 mmol) of melamine, 200 g (2.27 mol) of ethylene carbonate and 1.40 g (17.5 mmol) of aqueous sodium hydroxide solution (50% by weight) are introduced into a flask and heated to 170° C. with stirring. The mixture thus obtained is stirred at 170° C. until evolution of gas can no longer be observed. It is subsequently cooled to room temperature, and 102 g of water are added. A pH of 5 is adjusted using aqueous sulfuric acid (50% by weight). About 250 g of condensation product (D) are obtained, solids content: 48% [M_{w}=960 g/mol [M_{w}/Mₙ=3.6]

### Example NM4 Condensation Product (D)

### Reactants:

urea
ethylene carbonate
potassium carbonate
sulfuric acid

7.60 g (127 mmol) of urea, 200 g (2.27 mol) of ethylene carbonate and 1.5 g (10.9 mmol) of potassium carbonate are introduced into a flask and heated to 170° C. with stirring. The mixture thus obtained is stirred at 170° C. until evolution of gas can no longer be observed. It is subsequently cooled to room temperature, 125 g of water are added and a pH value of 5 is adjusted using aqueous sulfuric acid (50% by weight). 250 g of condensation product (D) are obtained. Solids content: 47% [M_{w}=1920 g/mol, M_{w}/Mₙ=4.8].

### Examples of Condensation Products (A) and Condensation Products (B)

### General Preliminary Remarks:

Solutions are always understood as meaning aqueous solutions if not expressly specified otherwise.

ppm always relates to parts by weight.

The molecular weight determinations are carried out using gel permeation chromatography (GPC):

Stationary phase: poly(2-hydroxymethacrylate) gel crosslinked with ethylene glycol dimethacrylate, obtainable commercially as HEMA BIO from PSS, Mainz, Germany.
Eluent: mixture of 30% by weight of tetrahydrofuran (THF), 10% by weight of acrylonitrile, 60% by weight of 1 molar NaNO₃ solution
Internal standard: 0.001% by weight of benzophenone, based on eluent
Flow: 1.5 ml/min
Concentration: 1% by weight in the eluent containing internal standard
Detection: UV/Vis spectrometrically at 254 nm
Calibration using polystyrene calibration part from PSS.
Mₙ: number-average molecular weight in [g/mol]
M_{w}: weight-average molecular weight in [g/mol]

For the determination of free formaldehyde, a flow injection apparatus according to Huber is employed, see Fresenius Z. Anal. Chem. 1981, 309, 389. The column chosen is a thermostatted reaction column 170×10 mm, filled with glass beads, which is operated at 75° C. The detector (continuous flow detector) is set at a wavelength of 412 nm. The procedure is as follows:

For the preparation of a reagent solution, 62.5 g of ammonium acetate are dissolved in 500 ml of distilled water, 7.5 ml of concentrated acetic acid and 5.0 ml of acetylacetone are added and filled up to 1000 ml with distilled water.

0.1 g of the condensation product to be investigated is weighed into a 10 ml volumetric flask, filled up to 10 ml with distilled water and the respective sample solution is obtained.

100 μl of sample solution in each case are added, mixed with reagent solution and a mean residence time of 1.5 minutes is set, which corresponds to a flow of 35 ml/min.

For the determination of the absolute values, the flow injection apparatus is calibrated with formaldehyde solutions of known content.

1. Preparation of reaction solutions
1.1 Preparation of reaction solution 1.1

### Reactants:

a) phenol,
b) concentrated sulfuric acid,
c) formaldehyde,
d) urea

### Procedure:

2.04 kg of phenol are introduced into a stirring apparatus and treated with 2.48 kg of concentrated sulfuric acid (96% by weight) for 20 minutes. Care is to be taken here that the temperature does not exceed 105° C. Subsequently, the reaction mixture is stirred at 100 to 105° C. for 2 hours and then diluted with 0.34 kg of water of 20° C. and cooled to 70° C. 2.06 kg of aqueous urea solution (68% by weight) are metered in, the temperature rising to 95° C.; subsequently the mixture is cooled to 75° C. 4.10 kg of aqueous formaldehyde solution (30% by weight) are added over a period of 90 minutes, care being taken that the temperature does not rise above 75° C. Subsequently, it is partially neutralized using 0.78 kg of aqueous sodium hydroxide solution (50% by weight), 0.30 kg of water are added, and the mixture is subsequently stirred for 30 minutes and cooled further. 1.36 kg of phenol are added at a temperature of 50° C. 1.14 kg of aqueous formaldehyde solution (30% by weight) are subsequently metered in at 50° C. over 20 minutes and the mixture is subsequently stirred for a further 30 minutes at 55° C. The final adjustment of concentration and pH is carried out by addition of 1.40 kg of sodium hydroxide solution (50% by weight) and 2.5 kg of water. 18.5 kg of reaction solution 1.1 are obtained containing 43% by weight of nonvolatile fractions.

The analysis of reaction solution 1.1 affords the following values:

sodium sulfate by IC (based on nonvolatile fractions): 6.8% by weight;
phenol by HPLC (based on nonvolatile fractions): 0.36% by weight;
4-phenolsulfonic acid by HPLC (based on nonvolatile fractions): 2.89% by weight;
free formaldehyde: 75 ppm, based on nonvolatile fractions.
Mₙ 890 g/mol, M_{w} 7820 g/mol, determined by GPC.

### 1.2 Preparation of Reaction Solution 1.2

### Reactants:

a) phenol,
b) concentrated sulfuric acid,
c) formaldehyde,

### Procedure:

2.75 kg of phenol are introduced into a stirring apparatus and treated with 1.48 kg of concentrated sulfuric acid (96% by weight) for 20 minutes. Care is to be taken here that the temperature does not exceed 105° C. Subsequently, the reaction mixture is stirred at 100 to 105° C. for 3 hours and then cooled to 50° C. 2.00 kg of aqueous formaldehyde solution (30% by weight) are added over a period of approximately one hour, care being taken that the temperature does not exceed 55° C. Subsequently, the mixture is stirred at 50 to 55° C. for 10 hours, then 1.80 kg of water are added and it is finally stirred at 95 to 100° C. for 4 hours. After cooling to room temperature, the final adjustment of concentration and pH is carried out by addition of aqueous sodium hydroxide solution (50% by weight) and water. 10.2 kg of reaction solution 1.2 are obtained containing 40% by weight of nonvolatile fractions.

The analysis of reaction solution 1.2 affords the following values:

sodium sulfate by IC (based on nonvolatile fractions): 15.4% by weight;
phenol by HPLC (based on nonvolatile fractions): 0.11% by weight;
4-phenolsulfonic acid by HPLC (based on nonvolatile fractions): 5.34% by weight;
free formaldehyde: 8 ppm, based on nonvolatile fractions.
Mₙ 1810 g/mol, M_{w} 9040 g/mol, determined by GPC.

### 1.3 Preparation of Reaction Solution 1.3

### Reactants:

a) phenol,
b) concentrated sulfuric acid,
c) formaldehyde,
d) urea

### Procedure:

2.04 kg of phenol are introduced into a stirring apparatus and treated with 2.48 kg of concentrated sulfuric acid (96% by weight) for 20 minutes. Care is to be taken here that the temperature does not exceed 105° C. Subsequently, the reaction mixture is stirred at 100 to 105° C. for 2 hours and then diluted with 340 g of water. 2.05 kg of urea solution (68% by weight) are metered in, care being taken that the temperature does not exceed 95° C. 3.60 kg of aqueous formaldehyde solution (30% by weight) are then added at 83 to 93° C. over a period of 1.5 hours. After a stirring time of 15 minutes, 800 g of aqueous sodium hydroxide solution (50% by weight) are added, care being taken that the temperature does not exceed 85° C., so that the pH is subsequently between 7.3 and 7.5. 11.3 kg of reaction solution 1.3 containing 47% by weight of non-volatile fractions are obtained.

The analysis of reaction solution 1.3 affords the following values:

sodium sulfate by IC (based on nonvolatile fractions): 10.3% by weight;
phenol by HPLC (based on nonvolatile fractions): 0.74% by weight;
4-phenolsulfonic acid by HPLC (based on nonvolatile fractions): 1.36% by weight;
free formaldehyde: 99 ppm, based on nonvolatile fractions.
Mₙ 1990 g/mol, M_{w} 17.020 g/mol, determined by GPC.
2. Treatment of reaction solutions by molecular size-dependent separation processes

The molecular size-dependent separation processes chosen are ultrafiltrations.

2.00 kg of a reaction solution diluted with demineralized water to 20% by weight of nonvolatile fractions are ultrafiltered with the aid of a ceramic tube module. In the course of the ultrafiltration, demineralized water is continuously added for the filtrate removed, such that the amount of liquid in the filtration system remains constant. Here, product 1.1 to 1.3 is in each case first separated into a high molecular weight (2.1 h to 2.3 h) and a medium to low molecular weight fraction. In a second step, the medium to low molecular weight fraction is separated into a low (2.1 n to 2.3n) and a medium molecular weight (2.1 m to 2.3 m) fraction. In each case, 8 kg of solution of condensation product 2.1 h to 2.3 h, 2.1 m to 2.3 m and 2.1 n to 2.3 n are obtained in each case containing 8 to 12% by weight of nonvolatile fractions.

### Filtration Conditions:

### Temperature: 50° C.

Ceramic tube module in stainless steel housing, manufacturer Tami, module length 250 mm, module diameter 10 mm, specified separation limits (molecular weight cut-off) 15 000 D (first step) and 8000 D (second step), filtration area 0.0094 m², 3 channels.
Membrane material: Tami CeRAM membrane from Tami.
Inlet pressure (feed) between 2.5 and 5 bar, outlet pressure (retentate) between 2.5 and 5 bar. Transmembrane pressure difference 2.5 to 5 bar.
Feed rate 500-900 I/h
Inflow velocity approximately 1-6 m/s
Permeate flow between 7 and 28 kg/(m²×h)
Filtration period 10 to 25 hours.

### Analytical Values of Condensation Product 2.1 n (Example B1):

M_{w} 1980 g/mol, M_{w}/Mₙ=2.9, determined by GPC;
sodium sulfate by IC 1.0% by weight, based on nonvolatile fractions;
phenol by HPLC <0.01% by weight, based on nonvolatile fractions;
4-phenolsulfonic acid by HPLC <0.6% by weight, based on nonvolatile fractions,
free formaldehyde: 53 ppm, based on nonvolatile fractions.

### Analytical Values of Condensation Product 2.1 h (Example A1):

M_{w} 9610 g/mol, Mₙ 1230 g/mol, determined by GPC;
sodium sulfate by IC<0.1% by weight, based on nonvolatile fractions;
phenol by HPLC <0.06% by weight, based on nonvolatile fractions;
4-phenolsulfonic acid by HPLC 0.12% by weight, based on nonvolatile fractions,
free formaldehyde: 82 ppm, based on nonvolatile fractions.

### Analytical Values of Condensation Product 2.2 n (Example B2):

M_{w} 2340 g/mol, M_{w}/Mₙ=3.0, determined by GPC;
sodium sulfate by IC 12.3% by weight, based on nonvolatile fractions;
phenol by HPLC <0.05% by weight, based on nonvolatile fractions;
4-phenolsulfonic acid by HPLC 0.22% by weight, based on nonvolatile fractions,
free formaldehyde: 1 ppm, based on nonvolatile fractions.

### Analytical Values of Condensation Product 2.2 h (Example A2):

M_{w} 14 220 g/mol, Mₙ 3110 g/mol, determined by GPC;
sodium sulfate by IC 3.4% by weight, based on nonvolatile fractions;
phenol by HPLC <0.05% by weight, based on nonvolatile fractions;
4-phenolsulfonic acid by HPLC 0.77% by weight, based on nonvolatile fractions,
free formaldehyde: 16 ppm, based on nonvolatile fractions.

### Analytical Values of Condensation Product 2.3 n (Example B3)

M_{w} 3240 g/mol, M_{w}/Mₙ=3.7, determined by GPC;
sodium sulfate by IC 8.8% by weight, based on nonvolatile fractions;
phenol by HPLC <0.05% by weight, based on nonvolatile fractions;
4-phenolsulfonic acid by HPLC 0.38% by weight, based on nonvolatile fractions,
free formaldehyde: 30 ppm, based on nonvolatile fractions.

### Analytical Values of Condensation Product 2.3 h (Example A3):

M_{w} 20 570 g/mol, Mₙ 6530 g/mol, determined by GPC;
sodium sulfate by IC 0.67% by weight, based on nonvolatile fractions;
phenol by HPLC <0.05% by weight, based on nonvolatile fractions;
4-phenolsulfonic acid by HPLC 0.10% by weight, based on nonvolatile fractions,
free formaldehyde: 135 ppm, based on nonvolatile fractions.

Formulation for pharmaceutical compositions according to the invention in the form of base creams BC4.1 to BC4.3

The following are mixed

| | | |
|---|---|---|
| Mixture according to the invention of | 1.0 | g |
| condensation products (e.g. a + D, fractionated) | | |
| Triglycerol diisostearate | 3.0 | g |
| Isopropyl palmitate | 2.4 | g |
| Hydrophobic base gel DAC | 24.6 | g |
| Potassium sorbate | 0.14 | g |
| Anhydrous citric acid | 0.07 | g |
| Magnesium sulfate heptahydrate | 0.5 | g |
| Glycerol 85% by weight | 5.0 | g |
| Purified water fill up to | 100.0 | g and stir |

Formulation for pharmaceutical compositions according to the invention in the form of nonionic, hydrophilic creams C4.1 to C4.3

The following are mixed

| | | |
|---|---|---|
| Mixture according to the invention of | 1.0 | g |
| condensation products | | |
| Isooctyl laurate/myristate | 10.0 | g |
| Nonionic emulsifying alcohols | 21.0 | g |
| Glycerol 85% by weight | 5.0 | g |
| Potassium sorbate | 0.14 | g |
| Anhydrous citric acid | 0.07 | g |
| Purified water fill up to | 100.0 | g and stir |

### Pharmacological Investigations:

α) Test for the inhibition of the enzyme human leucocyte elastase.

For this, the action of mixtures according to the invention on the enzymatic action of elastase is investigated. The enzyme is incubated together with a substrate (active compound mixture according to working examples) and measured at various concentrations of the substrate conversion. If not much substrate is reacted, this is proof of the fact that the enzymatic action is inhibited by the mixture according to the invention.

Concretely, the reaction of the synthetic enzyme substrate AAPV (N-methoxysuccinyl-Ala-Ala-Pro-Val-p-nitroanilide) by the enzyme human leucocyte elastase according to U. P. S. Mrowietz et al., Selective Inactivation of human neutrophil elastase by synthetic tannin. J. Invest. Dermatol. 1991, 97, 529-533 is investigated.
β) Inhibition of plasmin by mixture according to the invention

The protease plasmin is also effectively inhibited by mixtures according to the invention. According to recent investigations, plasmin is able to activate cytokines. Therefore the effect of inhibiting plasmin also has importance for the pathogenesis of herpes labialis. Thus plasmin can activate the growth factor TGFβ from its inactive form by cleavage of the “latency protein”.
γ) Determination of the antiviral action by the example of herpes simplex virus type 1 and human papillomavirus type 16

The action of mixtures according to the invention on the inhibition of virus replication is investigated in specific culture systems in each case by adding the substance to the target cells (Vero cells) simultaneously to the virus suspension. The process is described in more detail as follows.

### Process for the Determination of the Antiviral Activity

In the investigation, it is determined whether a substance has antiviral activity against appropriately defined viruses and what amount of antiviral substance is needed in order to cause a 50% reduction of virus replication.

The virus dilution for use is determined with the aid of an endpoint titration of the cultured virus isolated. In this titration, the amount of virus is determined at which 50% of the batches from the virus dilution are infected or not infected (=infectious dose 50%=TCID50/ml).

A dilution serious increasing by the factor two is prepared from the substance to be tested. A defined amount of virus is then added. The substance/virus mixture is added to monolayers of suitable cells (here Vero cells). After an incubation period dependent on the virus, an assessment of the virus-related cytopathogenic change (CPE) is carried out. For the determination of the results, staining by means of antibodies against the virus employed is added. Here, a percentage estimation of the CPE is carried out in comparison to the virus control, which is set at 100%. During the staining, a photometric analysis is carried out. By means of linear regression using a computer program the concentration is calculated at which a 50% reduction of the virus replication of patient isolates is caused (IC₅₀).

### Preparation and Division of the Cell Suspensions

### 1. General

the cell culture is trypsinized, homogenized and transferred to growth medium
about 50 μl of cell suspension per hole are prepared

### Titration of the Viruses

preparation of a 1:10 dilution series
50 μl of dilution in each case are pipetted per hole in the 8-fold batch into a plate prepared with cell suspension
depending upon the virus, incubate the plate in an incubator for a few days at 37° C. (three days with herpes simplex)
after the incubation period assess plates microscopically for CPE

### Test Procedure

prepare a 1:2 dilution series using the substance to be tested
In row 1 of the plate add 100 μl of medium to the cells (no substance and virus dilution)
In row 2 pipette 50 μl of medium (no substance dilution)
distribute substance dilution rows 3-12 in the 8-fold batch on the plate

### Test Analysis

After the end of the incubation period analyze the plate microscopically for CPE. The virus control corresponds to 100% here. For all substance dilutions, the extent of cell destruction is indicated as a percentage by comparison with the virus control. After visual analysis, staining preferably follows.
   Staining with Virus-Specific Antibodies
under the sterile workbench, aspirate supernatant from microtiter plates
fixation of the cells with acetone/methanol
aspirate liquid
dilution of the virus-specific antibody with blocking solution. 50 μl are employed per cavity. The optimum concentration for the antibody is determined by means of titration for each new batch. Incubation for 1 h at 37° C.
3× washing with wash buffer
biotinylated anti-IgG antibodies are diluted in wash buffer and 50 μl each are pipetted into each cavity. The optimum concentration for the antibody is determined by means of titration for each new batch.
incubation for 1 h at 37° C.
3× washing
the streptavidin/peroxidase conjugate is diluted in wash buffer and 50 μl per cavity is employed. The optimum concentration of the conjugate is determined by means of titration for each new batch.
incubation for 30 min at 37° C.
3× washing
5 μp of substrate solution are pipetted into each cavity
in the case of use of a soluble substrate, pipette 2 rows containing 50 μl of substrate (=substrate blank value) on a separate plate
incubation for 10 min at RT
for stopping in the case of soluble substrate, add 100 ml of 1 N sulfuric acid analysis photometrically at a wavelength of 450 nm and a reference wavelength of 630 nm
the analysis is carried out within one hour after the end of the test

### Calculation of the IC₅₀ Value

from the 8 individual values of the controls or of the substance determinations determine the respective mean value
subtract the substrate blank value from all values
in the case of the antiviral determination the value of the VK corresponds to 100%
for the individual values of the substance determinations, calculate the % value in relation to the respective control
employ determined % values in the computer program Calcusyn for Windows (Biosoft) and calculate the IC₅₀ value.

TABLE 1
Inhibition of leucocyte elastase and virus activity of polycondensates
against herpes simplex type 1 and human papillomavirus type 16

| | | | | Relative | | |
|---|---|---|---|---|---|---|
| | | | | inhibition | | |
| | | | | leucocyte | | |
| | | Molecular | | elastase | | |
| | | weight | Mw/ | polymer 1.3 = | | IC 50 |
| Example | Polycondensate | Mw | Mn | 100% | Virus | [μg/ml] |
| Example 1 | NM1 | 2850 | 11.3 | 0.35 | Herpes simplex | 48.5 |
| Example 2 | NM2 | 2640 | 8.8 | 0.45 | Herpes simplex | 31.7 |
| Example 3 | NM2 | 2640 | 8.8 | — | Human papilloma | Not |
| | | | | | | active |
| Example 4 | NM3 | 960 | 6.4 | 0.8 | Herpes simplex | 24.0 |
| Example 5 | NM3 | 960 | 6.4 | — | Human papilloma | 60.3 |
| Example 6 | 1.1 | 7820 | 8.8 | 0.85 | Herpes simplex | 18.5 |
| Example 6 | 1.3 | 17 020 | 8.6 | 1.0 | Herpes simplex | 14.6 |
| Example 7 | 1.3 | 17 020 | 8.6 | — | Human papilloma | 18.7 |
| Example 8 | A1 | 9610 | 4.3 | 2.0 | Herpes simplex | 8.4 |
| Example 9 | A3 | 20 570 | 3.2 | 1.8 | Herpes simplex | 9.0 |
| Example 10 | 2.1m | 3880 | 3.3 | 1.5 | Herpes simplex | 12.9 |
| Example 11 | B1 | 1980 | 2.9 | 1.1 | Herpes simplex | 13.0 |
| Example 12 | B3 | 3240 | 3.7 | 1.8 | Herpes simplex | 6.5 |
| Example 13 | A3 | 20 570 | 3.2 | — | Human papilloma | 10.4 |
| Example 14 | B3 | 3240 | 3.7 | — | Human papilloma | 15.4 |
| Example 15 | M1 | — | — | 3.8 | Herpes simplex | 2.8 |
| Example 16 | M2 | — | — | 4.7 | Herpes simplex | 2.0 |
| Example 17 | M3 | — | — | 5.9 | Herpes simplex | 1.4 |
| Example 18 | M4 | — | — | 9.8 | Herpes simplex | <1 |
| Example 19 | M5 | — | — | 7.6 | Herpes simplex | 1.6 |
| Example 20 | M6 | — | — | 10.9 | Herpes simplex | <1 |
| Example 21 | M1 | — | — | — | Human papilloma | 3.2 |
| Example 22 | M2 | — | — | — | Human papilloma | 6.8 |
| Example 23 | M3 | — | — | — | Human papilloma | 1.6 |
| Example 24 | M6 | — | | — | Human papilloma | <1 |

TABLE 2
Polymer/active compound mixtures M 1-M 6

| | Component I/ | Component II/ | Component III/ | Component IV/ |
|---|---|---|---|---|
| Poly- | fraction | fraction | fraction | fraction |
| mer | [% by weight] | [% by weight] | [% by weight] | [% by weight] |
| M1 | A1/60 | B1/40 | — | — |
| M2 | A1/60 | B1/30 | NM3/10 | — |
| M3 | A1/60 | B1/30 | NM3/5 | Mimosa*/5 |
| M4 | A3/60 | B3/40 | Epigal- | — |
| | | | locatechol | |
| | | | gallate**/5 | |
| M5 | A3/50 | B3/40 | NM3/10 | — |
| M6 | A3/65 | B3/20 | B1/15 | — |

*plant extract of mimosa, Silvachimica srl, Italy, S. Michele Mondovi
**plant extract of green tea

δ) Further investigations on the antiviral action

The action of mixtures according to the invention on the inhibition of virus replication is investigated in specific culture systems in each case. Accordingly, mixture according to the invention inhibits the replication of herpes simplex virus type 1 if the substance is added to the target cells (Vero cells) simultaneously to the virus suspension.
ε) Substanz P— and anti-IgE-induced histamine release from human mast cells

In the investigations carried out, an inhibition of the anti-IgE-induced histamine release was seen at a concentration of 1 μg of mixture according to the invention/ml of a Pipes buffer (aqueous MgCl₂/CaCl₂ solution, T. Zuberbier et al., Allergy 1999, 54, 898).

1-16. (canceled)

17: A mixture comprising
i) at least one condensation product (A) obtainable by reaction of
a1) at least one aromatic system or heteroaromatic system,
a2) at least one carbonyl compound,
a3) if appropriate at least one sulfonating agent and
a4) if appropriate at least one urea derivative,
where the condensation product (A) has an M_{w} value ≧9000 g/mol, and
ii) at least one tanning agent having an M_{w} value <3000 g/mol,
wherein the individual components of the mixture (i and ii)
a) are prepared separately by reaction of the respective starting materials and if appropriate are subjected to a molecular size-dependent separation process, whereupon the individual components are mixed to give the mixture, or
b) if the mixture contains at least one condensation product (A) and at least one condensation product (B), these two condensation products are prepared together and subsequently isolated by a molecular size-dependent separation process, whereupon the two isolated condensation products (A) and (B) are mixed to give the mixture where condensation product (B) is obtainable by reaction of
b1) at least one aromatic system or heteroaromatic system,
b2) at least one carbonyl compound,
b3) if appropriate at least one sulfonating agent and
b4) if appropriate at least one urea derivative,
where the condensation product (B) has an M_{w} value between 300 and 3000 g/mol.

18: The mixture according to claim 17, wherein the tanning agent having an M_{w} value ≦3000 g/mol is at least one synthetic tanning agent, selected from the condensation products B) to D) with
condensation product (B) according to claim 1
condensation product (C) obtainable by reaction of
c1) melamine or urea
c2) glyoxal, glyoxylic acid or an alkali metal salt thereof,
c3) if appropriate at least one aromatic compound having at least one phenolic hydroxyl group and
c4) if appropriate at least one condensable compound having a reactive nitrogen-containing group,
where the condensation product (C) has an M_{w} value between 300 and 3000 g/mol, condensation product (D) obtainable by reaction of
d1) at least one cyclic organic carbonate with
d2) at least one compound having at least two nucleophilic groups per molecule, chosen from sulfonic acid groups, hydroxyl groups, primary or secondary amino groups or mercapto groups,
where the condensation product (D) has an M_{w} value between 300 and 3000 g/mol.

19: The mixture according to claim 17, wherein
the component a1) contains at least one compound which is substituted by at least one hydroxyl group or
the component b 1) contains at least one compound which is substituted by at least one hydroxyl group or
the component c3) is present or
the component d2) contains at least one compound having at least one hydroxyl group as a nucleophilic group.

20: The mixture according to claim 17, wherein the condensation product (A) is obtainable by reaction of
a1) phenol,
a2) at least one aldehyde selected from formaldehyde, acetaldehyde and propionaldehyde
a3) if appropriate concentrated sulfuric acid
a4) if appropriate at least one urea derivative selected from urea, melamine, 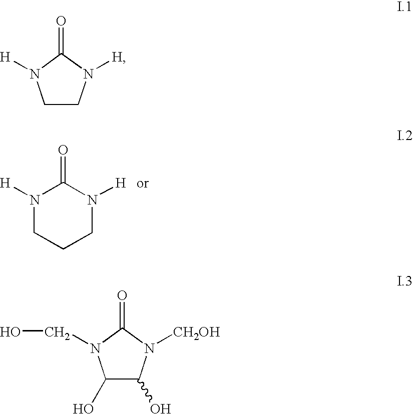
or
the condensation product (B) is obtainable by reaction of
b1) at least one compound selected from phenol and dihydroxydiphenylsulfone
b2) at least one aldehyde selected from formaldehyde, acetaldehyde and propionaldehyde
b3) if appropriate concentrated sulfuric acid
b4) if appropriate at least one urea derivative selected from urea, melamine, 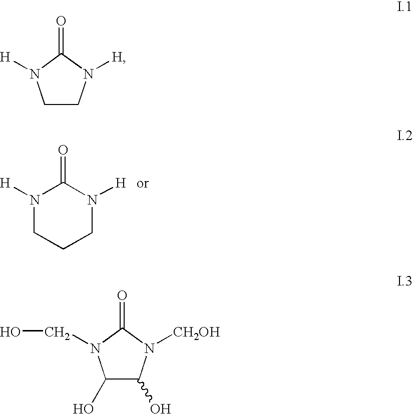
or
the condensation product (C) is obtainable by reaction of
c1) melamine or urea
c2) glyoxal or glyoxylic acid, and
c4) if appropriate amidosulfonic acid,
or
the condensation product (D) is obtainable by reaction of
d1) at least one compound selected from ethylene carbonate, propylene carbonate or mixtures thereof,
d2) at least one compound selected from melamine, biuret, dicyandiamide, amidosulfonic acid and 4,4′-dihydroxydiphenylsulfone.

21: The mixture according to claim 17, wherein, in the preparation of the condensation product (A), after reaction of the components a1) and a2) and if appropriate a3) and a4) an ultrafiltration is carried out with obtainment of the condensation product (A) having an M_{w} value of 10 000 to 100 000 g/mol.

22: The mixture according to claim 17, wherein the tanning agent having an M_{w} value ≦3000 g/mol is at least one plant tanning agent.

23: The mixture according to claim 22, wherein the plant tanning agent is a tannin or a gallic acid derivative.

24: The mixture according to claim 17, wherein the sum of components (i) at least 90% by weight related to the condensation products or tanning agents contained in the mixture.

25: The mixture according to claim 17 to be administered as a medicament.

26: A method for the prophylaxis or treatment of genital warts, cancer of the uterine cervix, allergic or nonallergic eczema, diaper rash, pruritus, inflammatory diseases, autoimmune diseases, rheumatism, melanomatous carcinomas, inflammations of the skin, herpes, chickenpox or AIDS, comprising administering to animals or humans in need thereof an efficacious amount of a condensation product according to claim 17 or a physiologically tolerable salt thereof.

27: The method according to claim 26, wherein herpes is herpes labilis or herpes simplex.

28: A method of treating animals or humans in need thereof with an antiviral agent, comprising administering an efficacious amount of a condensation product according to claim 17 or a physiologically tolerable salt thereof.

29: The method according to claim 28, wherein the antiviral agent is against human papillomaviruses, endogenous retroviruses, herpes viruses, HCMV viruses, HIV viruses, coronaviruses, flaviviruses, togaviruses or paramyxoviruses.

30: A pharmaceutical composition comprising an efficacious amount of at least one mixture according to claim 17 and a physiologically tolerable vehicle.

31: The pharmaceutical composition according to claim 30, where the pharmaceutical composition is present in the form of a pill, tablet, lozenge, granules, capsule, hard or soft gelatin capsule, aqueous solution, alcoholic solution, oily solution, syrup, emulsion, suspension, suppository, pastille, solution for injection or infusion, ointment, tincture, cream, lotion, powder, spray, of a transdermal therapeutic system, nasal spray, aerosol, aerosol mixture, microcapsule, implant, rod, patch or gel or in that the pharmaceutical composition according to the invention is a constituent of health care products such as sunscreen creams, nasal sprays, mouthwashes, toothpastes, plasters, (moist) wipes, washing lotions or shampoos.

32: A process for the production of the mixture according to claim 17, wherein the individual constituents of the mixture (at least one condensation product (A) and at least one tanning agent having an M_{w}. value ≦3000 g/mol) are prepared separately by reaction of the respective starting materials and if appropriate are subjected to a molecular size-dependent separation process, in which the individual components are mixed to give a mixture or, if the mixture contains at least one condensation product (A) and at least one condensation product (B), these two condensation products are prepared together and subsequently isolated by a molecular size-dependent separation process, in which they are mixed, if appropriate with further condensation products (A) or (B) or tanning agents having an M_{w} value ≦3000 g/mol, to give the mixture.

33: A method of disinfection employing a condensation product according to claim 17 as a disinfectant or constituent of a disinfectant in the hospital sector, toilets, washrooms, households, food production or in stables or cages of animals.

34: A disinfectant comprising at least one mixture according to claim 17.
